(19) United States
(12) Patent Application Publication
(10) Pub. No.: US 2021/0188905 A1
(43) Pub. Date: Jun. 24, 2021

(54) COMBINING BETA-DIPEPTIDES AND AMINO ACIDS FOR OPTIMAL NUTRITIONAL SUPPLEMENTATION

(71) Applicant: Cysal GmbH, Muenster (DE)

(72) Inventors: Ahmed Sallam, Muenster (DE); Martin Krehenbrink, Muenster (DE)

(73) Assignee: Cysal GmbH, Muenster (DE)

(21) Appl. No.: 17/263,130

(22) PCT Filed: Jul. 31, 2019
(86) PCT No.: PCT/EP2019/070583

§ 371 Date: Jan. 25, 2021

(30) Foreign Application Priority Data
Aug. 1, 2018 (EP) 18186879.5

(51) Int. Cl. C07K 5/072; A23L 33/18

(52) U.S. Cl. CPC C07K 5/06113; A61K 38/00; A23L 33/18

(57) ABSTRACT

The invention relates to a nutritional supplement comprising a combination of one or more β-aspartyl-containing dipeptides, or oligomers thereof, or salts thereof, wherein each of the β-dipeptides comprises β-L-aspartyl as a first amino acid residue and an amino acid selected from arginine, lysine, ornithine, and citrulline as the second amino acid residue, and the respective second amino acid(s) or salts thereof. The invention further relates to the use of the combination for nutritional supplementation and to the combination for use in amino acid therapy.

The invention relates to a nutritional supplement comprising a combination of one or more β-aspartyl-containing dipeptides, or oligomers thereof, or salts thereof, wherein each of the β-dipeptides comprises β-L-aspartyl as a first amino acid residue and an amino acid selected from arginine, lysine, ornithine, and citrulline as the second amino acid residue, and the respective second amino acid(s) or salts thereof. The invention further relates to the use of the combination for nutritional supplementation and to the combination for use in amino acid therapy.

### BACKGROUND OF THE INVENTION

Supplementation with amino acids is widely practiced for people under mental or physical stress or by certain subjects such as exercising sportsmen and body builders, often in doses high above the physiologically utilizable limits though. For example, the dosage for the amino acid arginine is often recommended by manufacturers to be 6-12 g per day. However, there is a natural limit to how much arginine the human body can take-up at one time. Human use data indicates that arginine levels in blood do not increase beyond an oral consumption of 2.5 g arginine. For example, intake of 5 g arginine results in the same blood levels as 2.5 g arginine. Also, large amounts of Arginine can cause adverse effects such as gastrointestinal cramps or diarrhea. Oral arginine supplements available today have two limitations: First, increasing arginine levels is difficult; an increase of the arginine available to the body, e.g. during intense workout phases, is difficult to achieve in practice due to the saturation problem and negative side effects related to the intake of large amounts of arginine. Second, a frequent administration is inconvenient; the exercising person needs to take arginine several times per day to get the daily dosage recommended by the manufacturer (4×1.5 g per day and higher).

On the other hand, WO2009/150252 discloses that β-dipeptides such as β-Asp-Arg, which are obtainable by enzymatic digestion of cyanophycin, are a potential amino acid-containing and arginine-containing supplement. However, WO2009/150252 is not providing any solution as to the above uptake limitation of amino acids such as that of arginine.

Furthermore, combinations of β-L-aspartyl dipeptides, where the second amino acid residue is selected from arginine, lysine, ornithine, glutamate, citulline and canavanine, with free amino acids and their use in nutritional or cosmetic compositions is known from WO2017/174398, WO2017/068149 and WO2017/162879. Again, the uptake limitation of amino acids such as arginine is not addressed in said references as the selection of the free amino acid is not connected with the second amino acid of the β-L-aspartyl dipeptide.

### SHORT DESCRIPTION OF THE INVENTION

It has now been found that certain β-L-aspartyl dipeptides, notably those known from WO2009/150252, which have arginine or its structurally related derivatives, for example, citrulline or ornithine as bound second amino acid residue, in combination with the respective individual (single) amino acids arginine, citrulline and ornithine, do provide an enhanced and prolonged uptake of these amino acids. It is believed that this effect is caused by different uptake mechanisms of the β-dipeptides versus single amino acids (two separate specialized uptake routes). Also after the separate uptake of both components, the dipeptide and the amino acid, each shows a different physiological behavior; other than the free amino acid component of the combination, the dipeptide component is resistant to the plasma enzymes involved in the metabolism of its constituting amino acids (an effect which is believed to be due to the β-peptide bond of the dipeptide). Thus, the combination of both components represents an ideal composition/method to provide a short term and wide availability (the single amino acid) as well as a long term and targeted delivery (via the dipeptide) of the constituting amino acids. The invention thus provides:

(1) a nutritional supplement comprising a combination of one or more β-aspartyl-containing dipeptides, or oligomers thereof, or salts thereof, wherein each of the β-dipeptides comprises β-L-aspartyl residue as a first amino acid residue which is bound to an amino acid selected from arginine, ornithine, and citrulline as the second amino acid residue, and the respective individual (hereinafter also referred to as “single” or “free”) second amino acid(s) or salts thereof;
(2) in a preferred embodiment of the nutritional supplement as defined in (1) above, the combination comprises: the dipeptide β-L-aspartyl-L-arginine, and free L-arginine, or salts thereof, or the dipeptides β-L-aspartyl-L-arginine and β-L-aspartyl-L-lysine, and free L-arginine, or salts thereof, and optionally free lysine or salts thereof;
(3) a combination as defined in (1) or (2) above for use in amino acid therapy;
(4) the use of the combination as defined in (1) or (2) above as an amino acid supplement, for human nutrition and sport nutrition; and
(5) a method for amino acid therapy or supplementation which comprises applying the combination as defined in (1) or (2) above to a subject in need of said therapy or supplementation.

### SHORT DESCRIPTION OF THE FIGURES

FIG. 1: Concentrations in whole blood after oral administration of 2.5 g (Δ) or 5 g (▪) of the dipeptide. Error bars represent standard errors of the mean.

FIG. 2: Areas under the curves for the concentrations in whole blood after oral administration of 2.5 g (Δ) or 5 g (▪) of the dipeptide shown in FIG. 1.

FIG. 3: Concentrations of the single amino acid component (here arginine) in whole blood after oral administration of 2.5 g (▪) or 5 g (Δ). Error bars represent standard errors of the mean.

FIG. 4: Concentrations of the dipeptide component (Δ) and the amino acid component (▪) in whole blood after oral administration of a combination of 2.5 g of each. Error bars represent standard errors of the mean.

FIG. 5: Arginine arginase control reaction (concentration in Mol %)

FIG. 6: Free arginine and dipeptide hydrolyses by arginase (concentration in %)

FIG. 7: Dipeptide treatment with different proteases for 24 h (concentration in %)

FIG. 8: Cleavage of the dipeptide (▪) and release of aspartic acid (Δ) by bovine liver extract at 37° C., 4-hour timescale.

### DETAILED DESCRIPTION OF THE INVENTION

The β-dipeptides or β-dipeptide oligomers of the combination of aspect (1) of the present invention are derived from cyanophycin, (also abbreviated CGP, Cyanophycin Granule Peptide) or a cyanophycin-like polymer by selective hydrolysis. In nature, and in addition to several heterotrophic bacteria, most cyanobacterial species (blue-green algae) accumulate the polypeptide CGP as a reserve material for carbon and nitrogen. CGP is accumulated in the early stationary growth phase of bacteria and is mostly composed of two amino acids, namely aspartic acid and arginine. One or more amino acids, which are structurally similar to arginine such as lysine, ornithine, glutamate, citrulline, and canavanine, may partially replace the arginine residue of CGP depending on the environmental/cultivation conditions.

Compared to chemically-synthesized dipeptides, CGP-dipeptides are natural and stereospecific (structurally homogeneous) substances that are produced from biomass in a biotechnological and environmentally-friendly way. The production of CGP dipeptides furthermore requires much less technological expense and effort, very little time, and significantly less financial effort. As the production process employs neither protecting groups nor harmful or environmentally unsafe solvents, the biocompatibility of these dipeptides is always ensured (Sallam et al. 2009. AEM 75:29-38).

Such CGP β-dipeptide compositions that are obtainable by the degradation/hydrolysis may be composed of a single type of β-dipeptides, or of a mixture of different β-dipeptides, or of a single type of β-dipeptide oligomers, or of a mixture of different β-dipeptide oligomers, or of mixtures of such β-dipeptides and β-dipeptide oligomers. It is however preferred that the β-dipeptides comprise amino acid residues selected from aspartate, arginine, lysine, and other amino acid residues present in CGP or CGP-like polymers. Particularly preferred is that the β-dipeptide is β-L-aspartyl-L-arginine.

A suitable CGPase for the CGP degradation is a CGPase from P. alcaligenes, particularly preferred from P. alcaligenes strain DIP1. Said CGPase (i) has a molecular weight of 45 kDa, an optimum temperature of 50° C., and an optimum pH range of 7-8.5 and degrades CGP into β-Asp-Arg; and/or (ii) is the P. alcaligenes DIP1 CGPase CphEₐₗ having been deposited with the DSMZ as DSM 21533, or is a mutant, derivative or fragment thereof capable of cleavage of CGP or CGP-like polymers into dipeptides.

The mutants, derivatives or fragments of the aforementioned native CGPase include fragments (having at least 50 consecutive amino acid residues of the native sequence, preferably N- and/or C-terminal truncation products, wherein up to 50 terminal amino acid residues are removed), derivatives (notably fusion products with functional proteins and peptides such as secretion peptides, leader sequences etc., and reaction products with chemical moieties such as PEG, alcohols, amines etc.) and mutants (notably addition, substitution, inversion and deletion mutants, having at least 80%, preferably at least 90%, most preferably at least 95% sequence identity with the native enzyme on the amino acid basis or wherein 1 to 20, preferably 1 to 10, consecutive or separated amino acid residues are added, substituted, inverted and/or deleted; for substitution mutants conservative substitution is particularly preferred), provided, however, that said modified CGPases have the enzymatic activity of the native CGPase.

The degradation process may be preceded by a step that provides the CGP or CGP-like polymer preparation, namely by culturing a prokaryotic or eukaryotic cell line. The producing cell line may be any cell line capable of producing the CGP or CGP-like polymer. It is preferred that the producing cell line is selected from Escherichia coli, Ralstonia eutropha, Acinetobacter baylyi, Corynebacterium glutamicum, Pseudomonas putida, yeast strains, and plant biomass. Particularly preferred producing cell lines are Ralstonia eutropha H16-PHB⁻4-Δeda (pBBR1MCS-2::cphA₆₃₀₈/edaH16) and E. coli DH1 (pMa/c5-914::cphA_{PCC6903}).

The above process may further comprise the steps of isolating, purifying and/or chemically modifying the CGP product obtained by cultivating the producing cell line. Such isolation, purification, chemical modification and separation may be effected by methods well established in the art.

It is however preferred that the CGP product obtained by cultivating the producing cell line is directly, i.e. without isolation or purification, subjected to degradation with the CGPase.

On the other hand, the degradation product may be purified and/or chemically modified. Again, such purification, separation, or chemical modification may be effected by methods well established in the art. It particularly includes the alkaline hydrolysis of the arginine residue in the β-Asp-Arg to citrulline and ornithine to give β-Asp-Cit and β-Asp-Orn as described in Example 2 below.

In the combination of aspect (1) each of the one or more β-dipeptides comprises β-L-aspartyl as a first amino acid residue, which is covalently bound to a second amino acid residue selected from arginine, ornithine and citrulline. In addition, the combination may contain structurally similar β-dipeptides, wherein the second amino acid residue is selected from lysine or canavanine. In any of these 3-dipeptides the second amino acid residue may be of L- or D-configuration. Thus, the dipeptides may have the formula I

(β-L-aspartyl-R)

and the dipeptide oligomers may have the formula II

(β-L-aspartyl-R)ₙ,

wherein R is independently selected from the amino acid residues defined herein-before and n is an integer of 2 to 150, preferably 2 to 30, most preferably 2 to 10. The combination of aspect (1) can further comprise two or more dipeptides as described above that are covalently bound together, and wherein the bound second amino acid residue of each dipeptide is independently selected, preferably selected from arginine, lysine, ornithine, citrulline, and canavanine. Most preferably the second amino acid residue is arginine or lysine. In another embodiment, one or more of the β-dipeptides are chemically modified. Such chemical modification includes phosphorylation, farnesylation, ubiquitination, gly-cosylation, acetylation, formylation, amidation, sumoylation, biotinylation, N-acylation, esterification, and cyclization.

Finally, both components, the β-aspartyl dipeptide(s) and the amino acid(s), are combined to obtain the desired final combination. This step can be performed by grinding both components in powder form together, for example, by standard “ball milling”. Whether the resulting combination of both components is a salt or a blend (mixture) or a mixture of both forms depends upon the ratio between the two components and the available humidity during this step. If the final combination is desired in liquid form, both components are to be combined by co-solving in a suitable liquid phase, e.g. water. The dosage form of the combination according to the present invention is not limited.

In a preferred embodiment, the nutritional supplement of aspect (1) and (2) comprises applicable daily doses from 0.01 to 25 g of β-dipeptide(s), or oligomer(s) or salt(s) thereof and from 0.01 to 25 g of the free basic amino acid or salt thereof, preferably from 1 to 15 g of β-dipeptide(s), or oligomer(s) or salt(s) thereof and from 1 to 15 g of the free basic amino acid or salt thereof, and most preferably from 2 to 5 g wt. % of β-dipeptides oligomer(s), or salt(s) thereof and from 2 to 5 g or 2 to 3 g of the free basic amino acid or salt thereof. In a further preferred embodiment, the combination of the nutritional supplement of aspect (1) and (2) comprises a molar ratio between the β-dipeptide(s), or salt(s) thereof and the amino acid in the combination, of from 99:1 to 1:99, preferably a ratio from 3:1 to 1:3, and most preferably a molar ratio of about 1:1, respectively.

Oligomers of the dipeptides include homomeric (i.e. composed of one β-dipeptide) and heteromeric (i.e. composed of two or more different β-dipeptides) structures, in which the β-dipeptide units are covalently attached to each other.

The β-dipeptidic products described above are highly stable under several conditions, and are suitable for being admixed with acceptable compounds conventionally used in nutritional supplements.

The product of aspects (1) and (2) may thus further comprise one or more free amino acids or salts thereof including but not limited to glutamine, histidine, tyrosine, BCAA, or tryptophan. The product may also further comprise one or more common nutritional ingredients including but not limited to creatine, whey protein, Taurine, Sustamine, or Carnosine.

The nutritional supplement of aspects (1) and (2) of the invention is particularly suitable for person in need of amino acid supplementation, including muscle growth and capacity, training/exercise duration, exercise tolerance, stimulation of growth hormone secretion, urea excretion, immunomodulation, weight control, supporting blood flow and cardiovascular functions, such as erectile dysfunction (ED) and regulation of blood pressure, nitrogen oxide (NO) stimulation and cell viability of human endothelial cells, NO stimulation and browning of adipocytes, proliferation and viability of skeletal muscle cells, and proliferation and viability of smooth muscle cells.

Aspect (3) of the invention pertains to the combination of aspects (1) and (2) for use in amino acid supplementation or therapy, in particularly for stimulation of growth hormone secretion, urea excretion, immunomodulation, supporting blood flow and cardiovascular functions, such as erectile dysfunction (ED) and regulation of blood pressure, nitrogen oxide (NO) stimulation and cell viability of human endothelial cells, NO stimulation and browning of adipocytes, proliferation and viability of skeletal muscle cells, and proliferation and viability of smooth muscle cells.

Aspects (4) and (5) of the invention relate to the use of the combination as defined in aspects (1) and (2) as an amino acids supplement, in food and human nutrition, sports nutrition, and to a method for amino acid therapy or supplementation which comprises applying the combination as defined in aspects (1) and (2) to a subject in need of said therapy or supplementation. In said use or method, the therapy and supplementation is preferably for muscle growth and capacity, training/exercise duration, exercise tolerance, stimulation of growth hormone secretion, urea excretion, immunomodulation, weight control, supporting blood flow and cardiovascular functions, such as erectile dysfunction (ED) and regulation of blood pressure, nitrogen oxide (NO) stimulation and cell viability of human endothelial cells, NO stimulation and browning of adipocytes, proliferation and viability of skeletal muscle cells, and proliferation and viability of smooth muscle cells.

The DIP1 CGPase CphEₐₗ was deposited by Westfälische Wilhelms-Universität Münster, Corrensstr. 3, 48149 Münster, Germany with the DSMZ-Deutsche Sammlung von Mikroorganismen und Zellkulturen GmbH, Inhoffenstr. 7b, 38124 Braunschweig, Germany as DSM 21533.

The invention will be further described in the following Examples, which are not to be construed as limiting the invention.

### EXAMPLES

### Example 1: Production of β-Aspartyl Dipeptides

CGP and the extracellular CGPase enzyme were produced via separate fermenta-tions before the final CGPase-catalyzed breakdown of CGP into dipeptides took place. A recombinant derivative of E. coli K12 harboring a commercial plasmid carrying the CGP synthetase gene (cphA) of Synechocystis sp. PCC6308 was used for the production of CGP in a 500 L fermentation, while the CGPase was produced with recombinant strain of Pichia pastoris harboring a genome integration of cphEₐₗ of the strain P. alcaligenes strain DIP1 having been deposited with the DSMZ as DSM 21533. CGP was then extracted from the produced biomass and purified. CGPase enzyme was applied as culture supernatant. The produced CGP and the CGPase were then combined under specific conditions, upon which the biopolymer was broken down into its constituent β-dipeptides. The β-L-aspartyl-L-arginine and β-L-aspartyl-L-lysine dipeptide fractions were then separated from the remainder of the reaction, analyzed for purity via HPLC, and finally dried to a powder (WO2009150252 and Sallam et al., AEM 75:29-38(2009)). For separating the two dipeptides, e.g. in order to obtain one of them in a pure form, a standard recrystallization procedures with alcohol can be applied as final step before drying the desired single recrystallized dipeptide.

### Example 2: Alkaline Hydrolysis of β-Asp-Arg to Produce β-Asp-Cit and β-Asp-Orn

By choosing appropriate conditions, the guanidino moiety of β-L-aspartyl-L-arginine can by hydrolyzed at alkaline pH to produce β-L-aspartyl-L-citrulline and β-L-aspartyl-L-ornithine without compromising the peptide bond.

β-L-Aspartyl-L-arginine was dissolved in water at concentrations up to the solubility limit at room temperature. The pH was then adjusted to a value between 12.5 and 13 using alkali or earth alkali hydroxide solution. The solution was then heated to the desired temperature. As higher temperatures accelerate the reaction, a convenient temperature was at or just below the boiling point of water. During the reaction, the pH was held constant by appropriate addition of alkaline solution. The reaction was complete when the pH remains stable without adjustment. The solution was then cooled to room temperature and the dipeptides were purified chromatographically. Typical conversion ratios are in excess of 95%. The proportion of β-L-aspartyl-L-citrulline to β-L-aspartyl-L-ornithine can be controlled by initial dipeptide concentration, pH value, and choice of alkaline solution.

### Example 3: Supplementation of β-Aspartyl Dipeptide Alone or in Combination with the Amino Acid Component

β-Aspartyl-arginine was administered orally either alone or in combination with arginine, and in varying doses. Levels of dipeptide in blood are then monitored over time. The substance used for the experiments is a white powder of β-aspartyl-arginine. The purity is >99% and was determined by HPLC-analysis.

Experimental procedure: The volunteers were three healthy males (age 41 to 51 years, 173-187 cm height, 80-85 kg weight, BMI around 25 kg/m²). The test substances (β-Asp-Arg dipeptide, arginine (as arginine aspartate salt), or a combination of the two) were given as a solution in 400 ml of water after overnight fasting. The volunteers fasted throughout the experiment. Blood was collected from the fingertip using a lancet device and blotted onto sample cards and levels of dipeptide and amino acids were determined by UPLC-MSMS by an external service provider (Labor Blessing, Singen Germany).

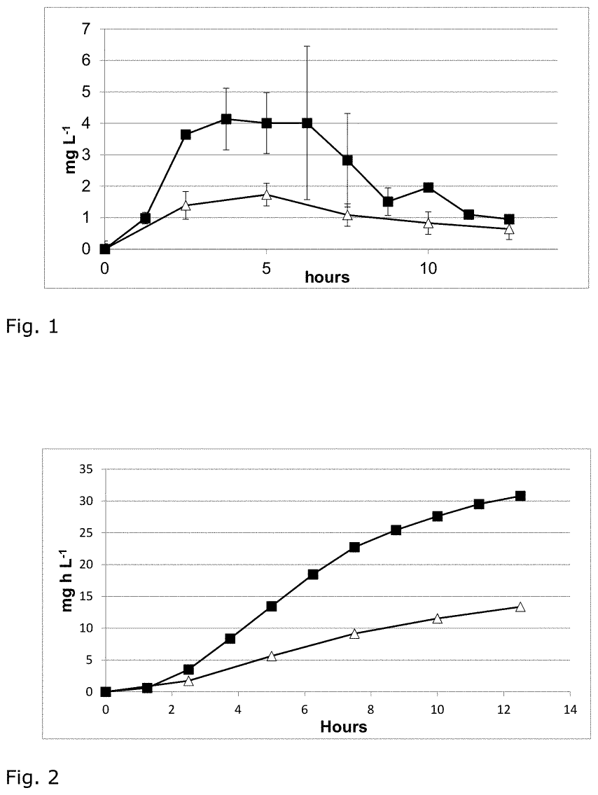

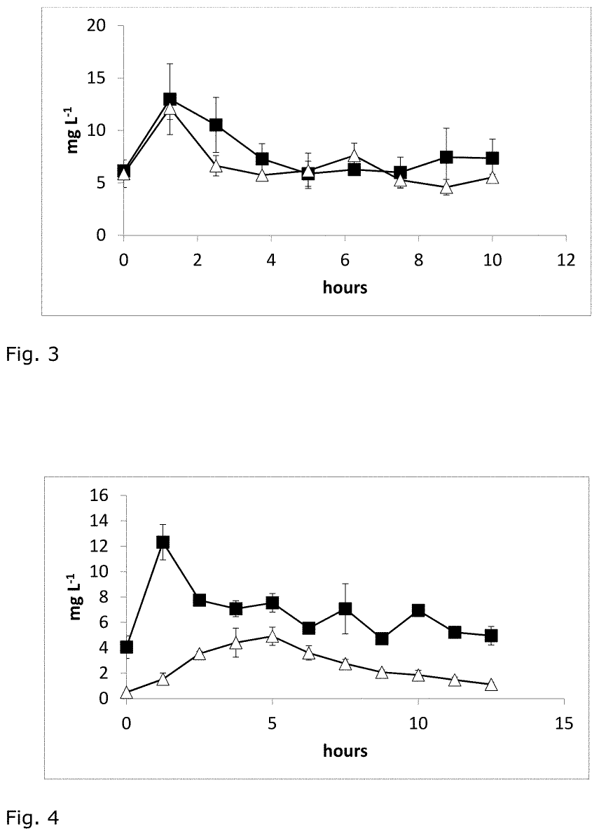

Results: Detection of β-aspartyl-arginine or arginine in the blood: In all three volunteers, the concentration of the dipeptide in whole blood increased over a period of about six hours, after which it began to decline and was still detectable for 12 h (FIG. 1). Free arginine was only detected at baseline levels. Doubling the oral dose from 2.5 g to 5 g approximately doubled the maximum concentration and also led to a doubling of the area under the curve (FIG. 2). In contrast, equimolar doses of free arginine (as arginine aspartate salt) led to a fast increase in blood concentration within two hours, but the concentrations returned to baseline within 4 h. The 5-g dose did not lead to substantially increased blood concentrations (FIG. 3). An area under the curve was not calculated as arginine is naturally present in the bloodstream.

Co-administration of β-aspartyl-arginine and arginine: Oral doses of a combination of 2.5 g each of β-aspartyl-arginine and arginine did not lead to a change in concentration profiles in blood compared to the profiles recorded for each of the two substances administered individually (FIG. 4).

Conclusion: Orally administered β-aspartyl-arginine is taken up into the bloodstream in the uncleaved dipeptide form. As no increase of free arginine was detected when the dipeptide was administered, cleavage rates in the intestine and the blood were probably negligible. The experiment also indicates that 2.5 g arginine is already at the blood saturation limit, as doubling the amount of substance did not lead to a relevant increase in arginine blood concentration. In contrast, doubling the oral dose of the dipeptide from 2.5 to 5 g led to an approximate doubling of the concentration in blood, suggesting that the saturation limit is not yet reached. Co-administration of both dipeptide and free arginine at the same time suggested that there is no interference in uptake between the two substances. It should be noted that this also implies different uptake routes, of which the different observed uptake kinetics are also likely to be a reflection. Thus, aspartyl-arginine is absorbed by the intestinal tract and passes into the bloodstream in the unhydrolyzed form.

### Example 4: Hydrolase Susceptibility of β-Aspartyl Dipeptide

Arginase catalyzes the final step of the urea cycle and converts L-arginine into L-ornithine und urea. The other tested enzymes (proteases) are able to cleave α-peptide bonds involving aspartate and/or arginine. The release of free amino acids or modified dipeptide after treatment with these enzymes is monitored by HPLC. The substance used for the experiments is a white powder of β-L-aspartyl-L-arginine. The purity is >99% and was determined by HPLC-analysis.

Procedure: Reaction conditions and specification for all tested enzymes are summarized in the table below.

| | | | | | | | Protease |
|---|---|---|---|---|---|---|---|
| | | | | | Endopro- | Proteinase | from |
| Reactions | | Clostri- | Chymo- | | teinase | N from | Rhizopus |
| (per ml) | Arginase | pain | trypsin | Trypsin | Arg-C | B. subtilis | sp. |
| 500 μg | 100 U | 10 U | 150 U | 187.5 U | 5 μl of | 28.4 U | 30 U |
| dipeptide | (1 mg) | | (100 μg) | (25 μg) | delivered | (4 mg) | (138 mg) |
| (or | | | | | solution | | |
| arginine | | | | | | | |
| for | | | | | | | |
| Arginase | | | | | | | |
| test) | | | | | | | |
| Reaction | 37° C. | 25° C. | 30° C. | 25° C. | 37° C. | temp. | temp. |
| temp. | | | | | | 37° C. | 37° C. |
| pH | 9.5 | 7.4 | 7.8 | 7.6 | 8.5 | 1-3 | 1-3 |
| Activa- | must be | must be | 100 mM | 67 mM | 100 mM | 67 mM | 67 mM |
| tion/ | activated | activated | Tris | sodium | Tris-HCl | sodium | sodium |
| Reaction | for 4 h. | for 3 h. | 10 mM | phos- | | phosphate | phos- |
| buffer | 0.05M | 10 mM | CaCl₂ | phate | | buffer | phate |
| | maleic acid | MOPS | | buffer | | | buffer |
| | with 0.05M | HCl | | | | | |
| | manganous | Buffer | | | | | |
| | sulfate | with 2.5 | | | | | |
| | | mM DTT, | | | | | |
| | | 1 mM | | | | | |
| | | CaCl₂ | | | | | |

### Results:

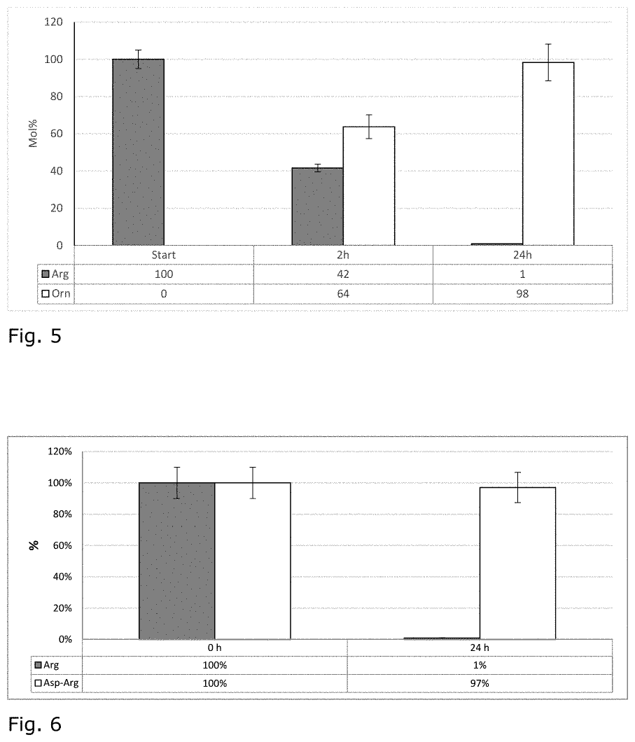

HPLC analysis—Arginase reactions: The control reaction (with free Arginine) for arginase showed that the enzyme is active and arginine is almost fully hydrolyzed to ornithine (FIG. 5). In contrast to the control reaction, it is no significant difference to the start concentration of dipeptides (FIG. 6).

HPLC analysis—Proteases: No significant difference to the start dipeptide concentration was observed by any of the tested proteases (FIG. 7).

Conclusion: β-Aspartyl-arginine is not susceptible to hydrolysis by any of the tested enzymes.

### Materials:

| Enzyme | Number | Supplier | Order number |
|---|---|---|---|
| Arginase (Bovine) | EC 3.5.3.1 | Alexis | ALX-201- |
| | | Bio/Sigma | 081-C020 |
| Endoproteinase | EC 3.4.21.35 | Sigma | P6056 |
| ArgC (mouse) | | | |
| Trypsin | EC 3.4.21.4 | Sigma | T1426-50MG |
| (Bovine pancreas) | | | |
| Chymotrypsin | EC 3.4.21.1 | Applichem | A4531 |
| (Bovine pancreas) | | | |
| Clostripain | EC 3.4.22.8 | Sigma | C0888-250UN |
| (Cl. histolyticum) | | | |
| Protease | CAS 9001-92-7 | Sigma | P0107 |
| (Rhizopus sp.) | | | |
| Proteinase N | CAS 116405- | Sigma | 82458 |
| (Bacillus subtilis) | 24-4 | | |

### Example 5: Cleavage of β-Aspartyl Dipeptides by Mammalian Enzymes

β-aspartyl dipeptides contain an isoaspartyl peptide bond instead of the α bond common in proteins. It is therefore resistant to cleavage by most common proteases and peptidases. While this resistance is an advantage in the gut and in the bloodstream as it prevents cleavage before reaching the target tissue, it does raise the question as to how the dipeptide is introduced into the metabolism. Specific cytoplasmic isoaspartases (also known as β-aspartyl peptidases) have been found in mammalian tissues that are capable of cleaving a large variety of β-aspartyl dipeptides and related compounds. Specificity is towards the β-aspartyl moiety, with the identity of the moiety bound to this residue being of little importance. The overall reaction can be summarized as:

β−Aspartyl−X+H₂O→Aspartic acid+X

The substance used for the experiments is a white powder of β-L-aspartyl-L-arginine. The purity is >99% and was determined by HPLC-analysis.

Experimental procedure: The liver is known to be highly metabolically active and has previously been shown to exhibit β-aspartyl dipeptidase activity (Dorer et al. 1968). Bovine liver purchased from a butcher was chosen as a model due to ready availability. Liver (50 g fresh weight) was homogenized using a Waring blender in four times its volume of ice-cold phosphate-buffered saline. Insoluble material was removed by centrifugation for 15 min at 9,000×g at 4° C. The supernatant (liver extract) was used immediately as a test solution.

Test setup: An aliquot of 900 μl of liver extract in a 1.5-ml polypropylene tube was placed into a heat block at 37° C. and allowed to heat up for 10 min. Then, a volume of 100 μl of a solution of 100 mM β-aspartyl-arginine phosphate-buffered saline was added to a give a final concentration of 10 mM. Samples of 100 μl were taken at 0, 1, 2, 3, and 4 h after addition of the dipeptide. Immediately after each sample was taken, it was added to a 1.5-ml screw-cap polypropylene tube containing 100 μl of 10% SDS in water and 700 μl of demineralized water. This tube was immediately heated to 100° C. for 10 min to stop any further enzyme activity. The tube was then cooled to room temperature and 100 μl of 10% KCl solution were added. The solution was then cooled on ice for at least 30 min to precipitate potassium dodecyl sulfate, which was sedimented by centrifugation at 13,000×g at 4° C. for 10 min along with any other insoluble debris. The samples were then diluted appropriately with demineralized water and analyzed by HPLC.

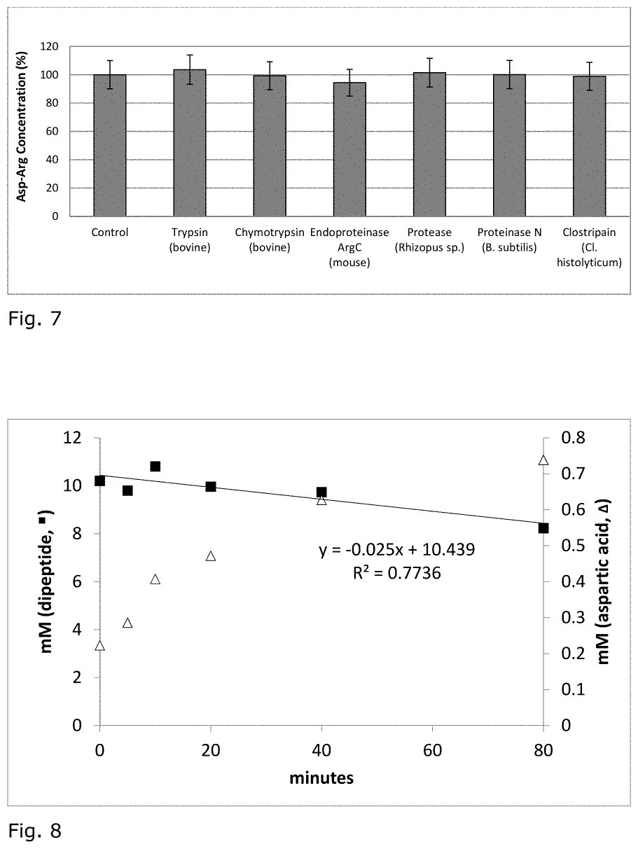

Results: A clear decrease of dipeptide and concomitant increase in free aspartic acid was observed. The hydrolysis rate appeared to slow as the experiment progressed, and the release of aspartic acid almost came to a standstill within two hours. As this may have been due to decreasing activity over time and side reaction sequestering the aspartate, the experiment was also repeated at a smaller timescale (FIG. 8). This found a higher overall activity and a better correlation of dipeptide hydrolysis and aspartate release rates. The activity corresponded to an activity of 2.5 mg of dipeptide being hydrolyzed per gram of liver tissue per hour. This equates to 0.065 U/mg protein, which compares well to the value found by Dorer et al. for rat liver extract using β-aspartyl-glycine as a substrate (0.028 U/mg). Thus β-aspartyl-arginine is cleaved by enzymes present in bovine liver. It is expected that β-aspartyl-arginine is cleaved to its constituting amino acids within the mammalian body, most probably also in other tissues where β-aspartyl peptidases are found.

1.-15. (canceled)

16. A nutritional or therapeutic supplement comprising a mixture of one or more β-aspartyl-containing dipeptides, or oligomers thereof, or salts thereof, wherein each of the β-dipeptides comprises β-L-aspartyl as a first amino acid residue which is bound to an amino acid selected from arginine, ornithine, and citrulline as the second amino acid residue, and the respective free second amino acid(s) or salts thereof.

17. The supplement of claim 16, wherein the amino acid component and the second amino acid of the β-aspartyl-containing dipeptide are of L- or D-configuration.

18. The supplement of claim 17, wherein the amino acid component and the second amino acid of the β-aspartyl-containing dipeptide are of L-configuration.

19. The supplement of claim 16, wherein the mixture further comprises one or more β-dipeptides, or oligomers thereof, or salts thereof, wherein each of the β-dipeptides comprise a β-L-aspartyl as a first amino acid residue and a bound second amino acid residue selected from lysine, and canavanine.

20. The supplement of claim 19, wherein
the second amino acid residue is lysine.

21. The supplement of claim 16, wherein the supplement comprises a mixture selected from the group consisting of
(i) the dipeptide β-L-aspartyl-L-arginine, and the amino acid arginine, or salts thereof;
(ii) the dipeptides β-L-aspartyl-L-arginine and β-L-aspartyl-L-lysine, and the amino acid arginine, and optionally the amino acid lysine, or salts thereof,
(iii) the dipeptide β-L-aspartyl-L-ornithine, and the amino acid ornithine, or salts thereof;
(iv) the dipeptide β-L-aspartyl-L-citrulline, and the amino acid citrulline, or salts thereof; and
(v) a mixture of any of the combinations described in (i) to (iv).

22. The supplement of claim 16, wherein
the oligomer comprises two or more covalently bound β-dipeptides.

23. The supplement of claim 16, wherein one or more of the β-dipeptides are chemically modified.

24. The supplement of claim 16, which comprises
a molar ratio between the β-dipeptide(s), or salt(s) thereof and the amino acid component in a range from about 99:1 to about 1:99.

25. The supplement of claim 24, which comprises a molar ratio in the range from about 3:1 to about 1:3, or a molar ratio of about 1:1.

26. The supplement of claim 16, which further comprises an applicable concentration of one or more free amino acids or salts thereof.

27. The supplement of claim 26, wherein the one or more free amino acids or salts thereof are selected from to the group consisting of glutamine, histidine, tyrosine, BCAA, and tryptophan.

28. The supplement of claim 16, which further comprises an applicable concentration of one or more components conventionally used in food or feed supplements.

29. The supplement of claim 28, wherein the one or more components conventionally used in food or feed supplements are selected from the group consisting of creatine, whey protein, Taurine, Sustamine, Carnosine, vitamins and minerals.

30. The supplement of claim 16, which is for a person in need of arginine supplementation, including muscle growth and capacity, training/exercise duration, exercise tolerance, stimulation of growth hormone secretion, urea excretion, immunomodulation, weight control, supporting blood flow and cardiovascular functions, such as erectile dysfunction (ED) and regulation of blood pressure, nitrogen oxide (NO) stimulation and cell viability of human endothelial cells, NO stimulation and browning of adipocytes, proliferation and viability of skeletal muscle cells, and proliferation and viability of smooth muscle cells.

31. The supplement of claim 16, which is for nutritional therapy.

32. The supplement of claim 16, which is an amino acid supplement for food, human nutrition and sport nutrition.

33. A method for amino acids therapy or supplementation which comprises applying a mixture of one or more β-aspartyl-containing dipeptides, or oligomers thereof, or salts thereof, wherein each of the β-dipeptides comprises β-L-aspartyl as a first amino acid residue which is bound to an amino acid selected from arginine, ornithine, and citrulline as the second amino acid residue, and the respective free second amino acid(s) or salts thereof, to a subject in need of said therapy or supplementation.

34. The method of claim 33, wherein the therapy and supplementation is for muscle growth and capacity, training/exercise duration, exercise tolerance, stimulation of growth hormone secretion, urea excretion, immunomodulation, weight control, supporting blood flow and cardiovascular functions, such as erectile dysfunction (ED) and regulation of blood pressure, nitrogen oxide (NO) stimulation and cell viability of human endothelial cells, NO stimulation and browning of adipocytes, proliferation and viability of skeletal muscle cells, and proliferation and viability of smooth muscle cells.
